(19) United States
(12) Patent Application Publication
(10) Pub. No.: US 2025/0268700 A1
(43) Pub. Date: Aug. 28, 2025

(54) Urinary Incontinence Device: A Non-Invasive Treatment for Urinary Incontinence

(71) Applicant: Haim Ahrony, Encino, CA (US)

(72) Inventor: Haim Ahrony, Encino, CA (US)

(21) Appl. No.: 18/733,589

(22) Filed: Jun. 4, 2024

Related U.S. Application Data
(63) Continuation of application No. 63/556,794, filed on Feb. 22, 2024.

(51) Int. Cl. A61F 2/00

(52) U.S. Cl. CPC A61F 2/0036; A61F 2/0009

(57) ABSTRACT

The Urinary Incontinence Device is a non-invasive treatment designed to integrate seamlessly with the bladder neck and urethra, facilitating unobstructed urine passage and regulating involuntary urine flow. The device features an elastic cap with a one-way valve mechanism, which opens upon bladder contraction to allow urine outflow and closes upon relaxation to prevent leakage. A closure mechanism mimics the natural urinary sphincter, and biocompatible materials minimize the risk of rejection. The device also includes a short elastic cylinder for additional support and stability. Optional adjustable components allow for customization to accommodate varying anatomical sizes and shapes. This innovative solution offers a reliable and discreet method for managing urinary incontinence, enhancing patient comfort and quality of life.

### BACKGROUND OF THE INVENTION

incontinence poses a widespread challenge affecting millions worldwide, significantly diminishing their quality of life. It arises from various factors, including age-related muscle weakening, pregnancy and childbirth, prostate issues, and physical disabilities. Beyond its physical effects, urinary incontinence can profoundly impact individuals' emotional well-being and social activities, leading to embarrassment, anxiety, and loss of confidence. Consequently, it may result in social withdrawal, decreased participation in activities, and feelings of isolation.

Current treatments for urinary incontinence vary from invasive surgical procedures to non-invasive methods, yet they often exhibit limitations. Invasive surgeries carry risks such as infection, prolonged recovery periods, and potential complications, while non-invasive methods may not consistently provide adequate relief. These limitations underscore the imperative for alternative solutions like the Urinary Incontinence Device, which offers a non-invasive option with fewer risks and greater effectiveness in managing urinary incontinence.

Among these alternatives, the Urinary Incontinence Device provides support to the bladder neck to regulate urine flow, seamlessly integrating with the natural anatomy of the bladder neck and urethra. Consisting of an elastic cap and a short elastic cylinder, these devices are surgically implanted with minimal risk of rejection or adverse reactions. Unlike invasive surgeries requiring hospitalization, general anesthesia, and extended recovery periods, the Urinary Incontinence Device can be implanted as an outpatient procedure, minimizing disruption to daily life for both patients and medical practitioners.

### SUMMARY OF THE INVENTION

The Urinary Incontinence Device described herein comprises several components aimed at seamlessly integrating with the natural anatomy of the bladder neck and urethra to facilitate urine passage and control involuntary urine flow. The key features of the device include:

### A. Elastic Cap

Engineered to seamlessly integrate with the natural bladder neck and urethra, aligning with their diameters to facilitate urine passage.
The elastic nature of the cap ensures a snug fit and optimal functionality.

### B. Closure Mechanism

Functionally replaces the natural urinary sphincter, aiding in controlling involuntary urine flow from the bladder to the urethra.
Designed to mimic the natural function of the urinary sphincter, providing precise control over urine flow.

### C. Biocompatible Materials

The closure mechanism further comprises biocompatible materials to minimize the risk of rejection or adverse reactions when implanted into the body.
Ensures compatibility with the patient's physiology.

### D. One-Way Valve Mechanism

Integrated into the cap, the one-way valve mechanism is configured to open upon bladder contraction to allow urine outflow and to close upon bladder relaxation to prevent leakage.
Ensures effective regulation of urine flow without compromising bladder function.

### E. Short Elastic Cylinder

Attached to the cap, the short elastic cylinder provides additional support and stability to the device.
Enhances the device's durability and performance.

### F. Adjustable Closure Mechanism (Optional)

Optionally, the device may include an adjustable closure mechanism to accommodate varying anatomical sizes and shapes.
Ensures a customized fit for each patient.

### G. Optional Valve Adjustment

Optionally, the device may include a valve that opens and closes based on changes in bladder pressure.
Provides further customization and control over urine flow regulation.

### CLAIMS SUPPORT

The features and functionalities described in the specification are consistent with the claims outlined in the patent application. The invention's design and operation align with the defined scope of protection sought for the Urinary Incontinence Device and method described herein.

### CONCLUSION

The Urinary Incontinence Device described herein offers a novel solution for addressing involuntary urine flow, providing effective control and regulation of urine flow through innovative design and functionality. With its seamless integration with the natural anatomy and customizable features, this device represents a significant advancement in the field of urinary incontinence treatment.

### DETAILED DESCRIPTION

### DESCRIPTION OF THE DRAWINGS

The accompanying drawings are included to provide a better understanding of the embodiments and are incorporated into and constitute a part of this specification. The drawings illustrate the embodiments and, together with the description, serve to explain the principles of the embodiments. Other embodiments and many of the intended advantages will be readily appreciated as they become better understood by reference to the following detailed description. The elements of the drawings are not necessarily to scale relative to each other.

FIG. 1: A view of the top cap of the Urinary Incontinence Device, demonstrating the embodiment of an incontinence treatment device including a closure one-way valve mechanism.

FIG. 2: A view of the bottom cap of the Urinary Incontinence Device, demonstrating the embodiment of an incontinence treatment device including a closure one-way valve mechanism.

FIG. 3: A view of the short elastic cylinder which will be attached to the cap, providing additional support and stability to the device, enhancing its durability and performance.

FIG. 4: A view of the Urinary Incontinence Device including the cap and short tube attached.

FIG. 5: A view of the anatomy of the bladder in the urinary system and how the Urinary Incontinence Device integrates with the natural anatomy and functionally replaces the natural urinary sphincter to control urine flow and treat incontinence.

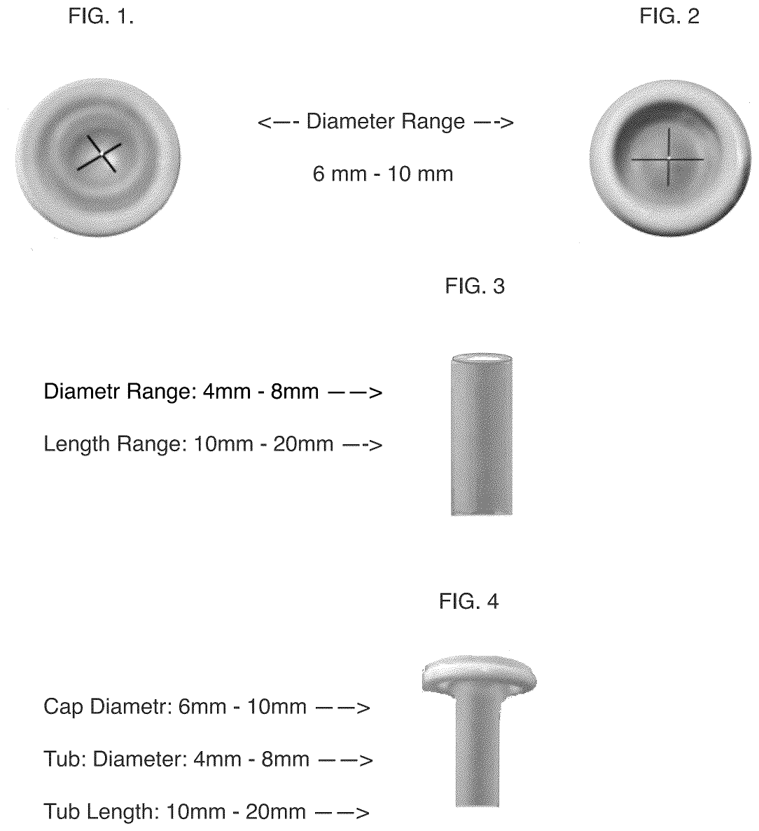

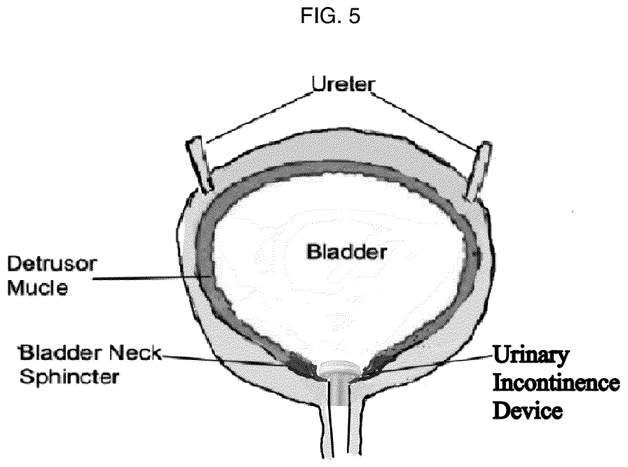

### INTRODUCTION

The present invention pertains to a Urinary Incontinence Device designed for the non-invasive treatment and prevention of urinary leakage, adaptable to both male and female anatomies.

### COMPONENTS AND DESIGN

The Urinary Incontinence Device comprises an elastic cap and a short elastic cylinder, both composed of biocompatible materials. These components are surgically implanted into the bladder neck and urethra, respectively, to regulate urine flow by mimicking the natural urinary sphincter's functionality.

### FUNCTIONALITY

The elastic cap and cylinder seamlessly integrate with the bladder neck and urethra, ensuring compatibility and facilitating unobstructed urine passage. The elastic cap is designed to compress to approximately 6 mm during insertion and expand up to 10 mm within the bladder neck to ensure a snug fit, preventing urine leakage. The short elastic cylinder, which provides additional support and stability, ranges from 10 mm to 20 mm in length and 4 mm to 8 mm in diameter. A closure mechanism within the device regulates urine flow, opening during bladder contraction and closing during relaxation to prevent leakage. Additionally, a one-way valve mechanism integrated into the cap dynamically facilitates urine outflow and prevents leakage based on bladder pressure changes.

### ADJUSTABLE COMPONENTS

The device includes adjustable components that can be fine-tuned during implantation to ensure a snug and comfortable fit for both male and female patients. The closure mechanism is designed to adapt to the specific anatomical features of the patient, providing effective urinary control regardless of gender.

### VARIATIONS AND EMBODIMENTS

The Urinary Incontinence Device may include variations to accommodate different anatomical sizes and shapes, considering the dimensions of the urethra in both male and female patients.

### Male Patients

For male patients, the device may feature an elastic cap with a diameter ranging from 6 mm to 10 mm and a short elastic cylinder with a length ranging from 15 mm to 20 mm and a diameter ranging from 6 mm to 8 mm. These dimensions are optimized to fit the longer and wider male urethra.

### Female Patients

For female patients, the device may feature an elastic cap with a diameter ranging from 6 mm to 10 mm and a short elastic cylinder with a length ranging from 10 mm to 15 mm and a diameter ranging from 4 mm to 6 mm. These dimensions are tailored to fit the shorter and narrower female urethra.

### ADVANTAGES

Compared to invasive surgeries, the Urinary Incontinence Device offers non-invasive outpatient implantation, minimizing hospitalization, anesthesia, and recovery periods. This feature enhances patient comfort and convenience while effectively managing urinary incontinence.

1. A urinary incontinence treatment device, comprising:
An elastic cap with a diameter range from 6 mm to 10 mm, engineered to seamlessly integrate with the natural bladder neck and urethra
A closure mechanism functionally replacing the natural urinary sphincter, aiding in controlling involuntary urine flow from the bladder to the urethra
Said closure mechanism further comprising biocompatible materials to minimize the risk of rejection or adverse reactions
A one-way valve mechanism integrated into the cap, said valve configured to open upon bladder contraction to allow urine outflow and to close upon bladder relaxation to prevent leakage.
A short elastic cylinder with a length range from 10 mm to 20 mm and diameter range from 4 mm to 8 mm attached to the cap to provide additional support and stability.
Optionally, an adjustable closure mechanism to accommodate varying anatomical sizes and shapes.
Optionally, a valve that opens and closes based on changes in bladder pressure.

2. The device of claim 1, wherein the closure mechanism mimics the natural function of the urinary sphincter.

3. The device of claim 1, further comprising:
A cap incorporating the one-way valve mechanism and the short elastic cylinder.

4. The device of claim 2, further comprising:
A cap incorporating the one-way valve mechanism and the short elastic cylinder.

5. A method for treating urinary incontinence, comprising:
Implanting the device of any one of claims 1-4 into a patient
Configuring the device to replace the natural urinary sphincter
Allowing the device to regulate urine flow by mimicking the natural function of the urinary sphincter and utilizing the one-way valve mechanism
Optionally, adjusting the pressure required to open the one-way valve mechanism based on the patient's needs
Optionally, performing the implantation process as an outpatient procedure
Optionally, implanting the device without the need for general anesthesia.
Optionally, minimizing disruption to the patient's daily activities post-implantation.

6. The device of any one of claims 1-4, wherein in the event of device dislodgment or failure, the reversal procedure is simple and outpatient, minimizing the need for additional invasive surgeries, reducing the risk of tissue erosion through the urethra and bladder, mitigating the potential for urinary retention, infection, and alleviating pain or soreness typically associated with other products.
